(19) United States
(12) Patent Application Publication
(10) Pub. No.: US 2025/0381161 A1
(43) Pub. Date: Dec. 18, 2025

(54) COMPOSITIONS AND METHODS FOR TREATING HEARING AND OCULAR DISORDERS

(71) Applicant: Anida Pharma Inc., Cambridge, MA (US)

(72) Inventor: Per Gjorstrup, Cambridge, MA (US)

(21) Appl. No.: 18/877,765

(22) PCT Filed: Jun. 21, 2023
(86) PCT No.: PCT/US23/25888

§ 371 Date: Dec. 20, 2024

Related U.S. Application Data
(60) Provisional application No. 63/353,948, filed on Jun. 21, 2022.

(51) Int. Cl. A61K 31/202; A61K 45/06; A61K 47/34

(52) U.S. Cl. CPC A61K 31/202; A61K 45/06; A61K 47/34

(57) ABSTRACT

The present invention relates to pharmaceutical compositions of docosahexaenoic acid (DHA) analogs for treating hearing and ocular disorders.

### CROSS-REFERENCE TO RELATED APPLICATIONS

This application claims the benefit of priority to U.S. Provisional Patent Application No. 63/353,948, filed Jun. 21, 2022, the entire contents of which are incorporated herein by reference.

### BACKGROUND OF THE INVENTION

There is a need to identify new drug compositions for sensory organ, eye and ear, dysfunction, with a focus on the retina and the inner ear. To efficiently deliver drug to the retina, the inner ear blood-retina-barrier or blood-inner ear-barrier must be overcome, which are evolutionary developed for protective purposes. To overcome these barriers to achieve effective drug concentrations in the target organ on systemic drug administration, high doses must be delivered and with risk of both adverse events and not reaching target drug concentrations. Alternatively, formulated drug can be locally delivered by injection, but oft-repeated injections are clinically impractical. Formulations need to be identified that following injection of drug product will allow for sustained release of drug over a longer period of time. The characteristic of such formulations to meet clinical needs will depend on the physiochemical properties of the drug to be delivered.

Accordingly, new formulations and methods for treating sensory organ dysfunction are needed.

### SUMMARY OF THE INVENTION

The present invention provides for an formulation, to over a defined period of time, deliver a therapeutically effective amount of a compound of Formula I, II, III or IV:

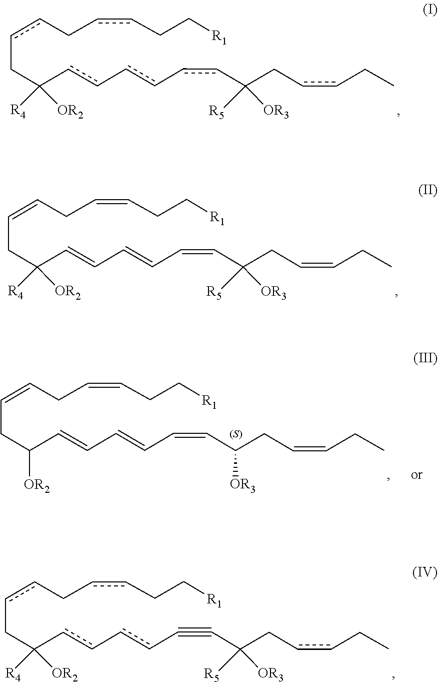

or a pharmaceutically acceptable salt, ester, or prodrug thereof. In Formula I, II, III, or IV:
[symbol] represents a cis or trans bond;
R₁ is —C(O)ORₐ, —C(O)NR_{b}R_{c}, —C(O)H, —C(NH)NR_{b}R_{c}, —C(S)H, —C(S)ORₐ. —C(S)NR_{b}R_{c}, or —CN;
R₂ and R₃ are each independently H or a protecting group;
R₄ and R₅ are each independently H, halo, unsubstituted or substituted C₁-C₆ alkyl, unsubstituted or substituted C₃-C₁₀ carbocycle, unsubstituted or substituted heterocycle comprising one or two 3-, 4-, 5- or 6-member rings and 1-4 heteroatoms selected from N, O and S,
Rₐ is a protecting group or —T₁-Q₁;
R_{b} and R_{c} are each independently a protecting group or -T₁-Q₁, or R_{b} and R_{c}, together with the nitrogen atom to which they are attached, form an unsubstituted or substituted heterocycle comprising one or two 5- or 6-member rings and 1-4 additional heteroatoms selected from N, O and S;
T₁ is a bond or unsubstituted or substituted C₁-C₆ alkyl linker; and
Q₁ is H, hydroxyl, halogen, unsubstituted or substituted C₁-C₆ alkyl, unsubstituted or substituted C₁-C₆ alkoxy, unsubstituted or substituted 2-6 membered heteroalkyl, unsubstituted or substituted C₃-C₁₀ carbocycle, or unsubstituted or substituted heterocycle comprising one or two 3-, 4-, 5- or 6-member rings and 1-4 heteroatoms selected from N, O and S.

In some embodiments, the formulation is slow-release. In some embodiments, the formulation is injectable.

The present invention also provides a method for treating sensory organ dysfunction by administering to a subject in need thereof a therapeutically effective amount of a pharmaceutical composition comprising a compound of Formula I, II, III, or IV, or a pharmaceutically acceptable salt, ester, prodrug, metabolite, analog or derivative thereof, in combination with a pharmaceutically acceptable excipient, such that sensory organ dysfunction is treated.

Preferred compounds and pharmaceutical compositions include sodium (4Z,7Z, 10R, 11E, 13E, 15Z, 17S, 19Z)-10, 17-dihydroxydocosa-4,7, 11, 13, 15, 19-hexaenoate and (4Z,7Z,10R,11E,13E, 15Z, 17S, 19Z)-10, 17-dihydroxydocosa-4,7,11, 13, 15, 19-hexaenoic acid.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a plot of the release of compound 1 formulated in an artificial tear medium over 24 hours.

FIG. 2 is a plot of the release of compound 1 formulated in the PEG-PCL-PEG co-polymer.

FIG. 3 is a plot showing liquid to semi-solid temperature-dependent conversion.

### DETAILED DESCRIPTION OF THE INVENTION

### 1. Pharmaceutical Compositions of Docosahexaenoic Acid (DHA) Analogs

The present invention provides a method by administering to a subject a DHA analog and its pharmaceutical composition for use in the treatment of sensory organ dysfunction.

Representative compounds (DHA analogs) useful for the method of the present invention include compounds listed in Table 1.

TABLE 1

| Compound | | |
|---|---|---|
| Number | Structure | Name |
| 1 | 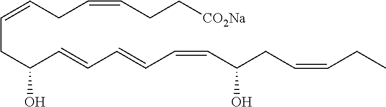 | Sodium (4Z,7Z,10R,11E,13E,15Z,17S, 19Z)-10,17-dihydroxydocosa- 4,7,11,13,15,19-hexaenoate |
| 2 | 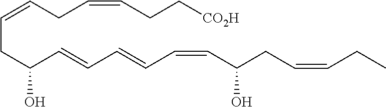 [SMILES: CC/C=C\C[C@H](O)\C=C/C=C/C=C/[C@H](O)C/C=C\C/C=C\CCC(=O)O] | (4Z,7Z,10R,11E,13E,15Z,17S, 19Z)-10,17-dihydroxydocosa- 4,7,11,13,15,19-hexaenoic acid |
| 3 | 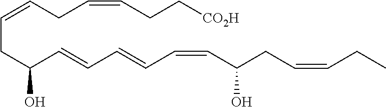 [SMILES: CC/C=C\C[C@H](O)\C=C/C=C/C=C/[C@@H](O)C/C=C\C/C=C\CCC(=O)O] | (4Z,7Z,10S,11E,13E,15Z,17S, 19Z)-10,17-dihydroxydocosa- 4,7,11,13,15,19-hexaenoic acid |
| 4 | 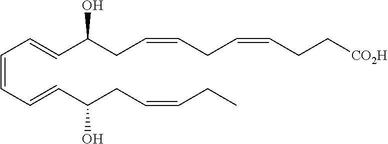 [SMILES: CC/C=C\C[C@H](O)/C=C/C=C\C=C\[C@@H](O)C/C=C\C/C=C\CCC(=O)O] | (4Z,7Z,10S,11E,13Z,15E,17S, 19Z)-10,17-dihydroxydocosa- 4,7,11,13,15,19-hexaenoic acid |
| 5 | 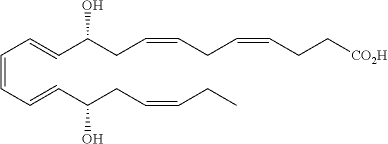 [SMILES: CC/C=C\C[C@H](O)/C=C/C=C\C=C\[C@H](O)C/C=C\C/C=C\CCC(=O)O] | (4Z,7Z,10R,11E,13Z,15E,17S, 19Z)-10,17-dihydroxydocosa- 4,7,11,13,15,19-hexaenoic acid |
| 6 | 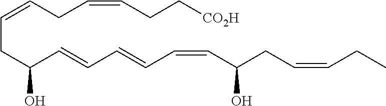 [SMILES: CC/C=C\C[C@@H](O)\C=C/C=C/C=C/[C@@H](O)C/C=C\C/C=C\CCC(=O)O] | (4Z,7Z,10S,11E,13E,15Z,17R, 19Z)-10,17-dihydroxydocosa- 4,7,11,13,15,19-hexaenoic acid |
| 7 | 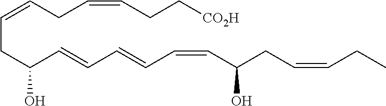 [SMILES: CC/C=C\C[C@@H](O)\C=C/C=C/C=C/[C@H](O)C/C=C\C/C=C\CCC(=O)O] | (4Z,7Z,10R,11E,13E,15Z,17R, 19Z)-10,17-dihydroxydocosa- 4,7,11,13,15,19-hexaenoic acid |
| 8 | 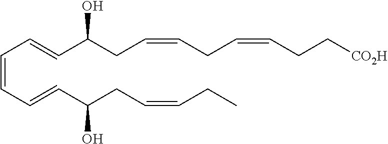 [SMILES: CC/C=C\C[C@@H](O)/C=C/C=C\C=C\[C@@H](O)C/C=C\C/C=C\CCC(=O)O] | (4Z,7Z,10S,11E,13Z,15E,17R, 19Z)-10,17-dihydroxydocosa- 4,7,11,13,15,19-hexaenoic acid |
| 9 | 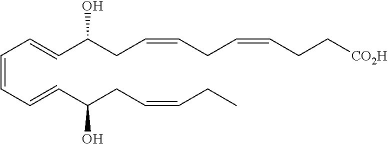 [SMILES: CC/C=C\C[C@@H](O)/C=C/C=C\C=C\[C@H](O)C/C=C\C/C=C\CCC(=O)O] | (4Z,7Z,10R,11E,13Z,15E,17R, 19Z)-10,17-dihydroxydocosa- 4,7,11,13,15,19-hexaenoic acid |
| 10 | 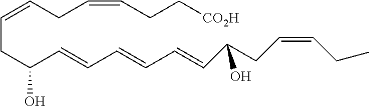 [SMILES: CC/C=C\C[C@H](O)/C=C/C=C/C=C/[C@H](O)C/C=C\C/C=C\CCC(=O)O] | (4Z,7Z,10R,11E,13E,15E,17S, 19Z)-10,17-dihydroxydocosa- 4,7,11,13,15,19-hexaenoic acid |
| 11 | 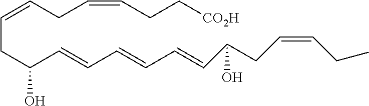 [SMILES: CC/C=C\C[C@@H](O)/C=C/C=C/C=C/[C@H](O)C/C=C\C/C=C\CCC(=O)O] | (4Z,7Z,10R,11E,13E,15E,17R, 19Z)-10,17-dihydroxydocosa- 4,7,11,13,15,19-hexaenoic acid |
| 12 | 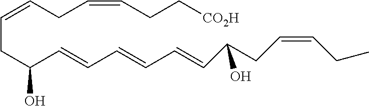 [SMILES: CC/C=C\C[C@H](O)/C=C/C=C/C=C/[C@@H](O)C/C=C\C/C=C\CCC(=O)O] | (4Z,7Z,10S,11E,13E,15E,17S, 19Z)-10,17-dihydroxydocosa- 4,7,11,13,15,19-hexaenoic acid |
| 13 | 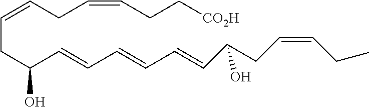 [SMILES: CC/C=C\C[C@@H](O)/C=C/C=C/C=C/[C@@H](O)C/C=C\C/C=C\CCC(=O)O] | (4Z,7Z,10S,11E,13E,15E,17R, 19Z)-10,17-dihydroxydocosa- 4,7,11,13,15,19-hexaenoic acid |
| 14 | 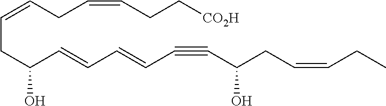 [SMILES: CC/C=C\C[C@H](O)C#C/C=C/C=C/[C@H](O)C/C=C\C/C=C\CCC(=O)O] | (4Z,7Z,10R,11E,13E,17S,19Z)- 10,17-dihydroxydocosa- 4,7,11,13,19-pentaen-15-ynoic acid |

The subject compound may be purified, e.g., substantially separated from other compounds or isomers that are present in a cellular environment where resolvins/protectins are produced or that are present in crude products of synthetic chemical manufacturing processes. In certain embodiments, a purified compound is contaminated with less than 25%, less than 15%, less than 10%, less than 5%, less than 2%, less than 1%, or less than 0.1% of cellular components (proteins, nucleic acids, carbohydrates, etc.), chemical byproducts, reagents, and starting materials, and the like. The addition of pharmaceutical excipients, other active agents, or other pharmaceutically acceptable additives is not understood to decrease the purity of a compound as this term is used herein.

The present invention provides methods for therapeutic compositions combining any of compounds listed in Table 1 imbedded with crosslinked polymers to achieve controlled release of the compound over a defined period of time.

A compound of the present invention is contaminated with less than 25%, less than 15%, less than 10%, less than 5%, less than 2%, less than 1%, or less than 0.1% of other resolvins and/or other isomers of the compound.

Therapeutic compositions may further include drug combinations with a compound in Table 1 and a second, or several, active compounds.

Said therapeutic compositions will be suitable for injection into the middle ear for release of drug to reach the inner ear; for injection into the vitreous, subsclearally, or subconjunctivally with the intent of drug release to a desired ocular target tissue, preferably the retina.

Crosslinked polymers will be biocompatible and biodegradable.

Crosslinked polymers will be thermosensitive meaning the polymers will exist as a clear solution at ambient temperature but when the temperature is raised above the LCST (lower critical solution temperature), which is preferably about body temperature, the polymers will interact to form a gel, suspension, or emulsion.

Crosslinked polymer solutions will be injectable to any target anatomical location using a 27-gauge injection needle, or finer.

A polymer with the desired crosslinking properties may be composed of any of the following:
a. PEG: polyethylene glycol;
b. PLC: poly (ε-caprolactone);
c. PGA: polyglycolic acid;
d. PLGA: poly (lactic-co-glycolide)

Any of PEG, PLC, PGA and PLGA may be combined in different sequence and of different compositions and as tri-block or penta-block polymers.

Any of PEG, PLC, PGA and PLGA may have an average molecular weight of 100-2,000 Da, the molecular weight further specified as to their position in a tri- or penta-block sequence.

A tri-block polymer may have a sequence of, but not limited to, PEG-PLC-PEG, PLC-PEG-PLC, or PLGA-PEG-PLGA.

A tri-block may be further combined with a poloxamer (Pluronic) which has a composition of poly (ethylene oxide)-poly (propylene oxide)-poly (ethylene oxide) (PEO-PPO-PEO).

A penta-block may have sequence of, but not limited to, PGA-PLC-PEG-PLC-PGA.

Tri-block or penta-block polymers will be dissolved in a physiologically acceptable liquid, which is preferably a buffer. Typically, the concentration of polymer is 5-75 wt %, and more preferably 10-50 wt %.

Crosslinked polymer compositions may also comprise nanoparticles containing a compound selected from Table 1.

Specifically, a nanoparticle may be composed by encapsulation of the compound with PLGA.

In another embodiment, a nanoparticle is formed by nanomicell formation to entrap the compound and may be formed with hydrogenated Castor oil (HCO) and Octoxynol (OC), including HCO40 and OC40, but different forms of HCO and OC in different combinations may also be suitable.

The therapeutic composition will contain at least 10% w/w of the compound preferably greater than 20% w/w of the active agent.

In certain embodiments, the controlled release pharmaceutical composition of the invention comprises compound 1. In further embodiments, the composition further comprises poly (ε-caprolactone) and poly (ethylene glycol) methyl ether (MPEG). In certain embodiments, compound 1 is present in an amount of about 0.01% w/v to about 1% w/v, preferably about 0.1% w/v. In some embodiments, the copolymer is present in an amount of about 10%-50% w/v, 20%-40% w/v, more preferably 25%-35% w/v, or about 30% w/v.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

“Heteroalkyl” groups are alkyl groups, as defined above, that have an oxygen, nitrogen, sulfur or phosphorous atom replacing one or more hydrocarbon backbone carbon atoms.

As used herein, the term “cycloalkyl”, “C₃, C₄, C₅, C₆, C₇ or C₈ cycloalkyl” or “C₃-C₈ cycloalkyl” is intended to include hydrocarbon rings having from three to eight carbon atoms in their ring structure. In one embodiment, a cycloalkyl group has five or six carbons in the ring structure.

The term “substituted alkyl” refers to alkyl moieties having substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)).

Unless the number of carbons is otherwise specified, “lower alkyl” includes an alkyl group, as defined above, having from one to six, or in another embodiment from one to four, carbon atoms in its backbone structure. “Lower alkenyl” and “lower alkynyl” have chain lengths of, for example, two to six or of two to four carbon atoms.

As used herein, “alkyl linker” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl linker is intended to include C₁, C₂, C₃, C₄, C₅ and C₆ alkyl linker groups. Examples of alkyl linker include, moieties having from one to six carbon atoms, such as, but not limited to, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂—), n-pentyl (—CH₂CH₂CH₂CH₂CH₂—), s-pentyl (—CHCH₃CH₂CH₂CH₂—) or n-hexyl (—CH₂CH₂CH₂CH₂CH₂CH₂—).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), branched alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched alkenyl group has six or fewer carbon atoms in its backbone (e.g., C₂-C₆ for straight chain, C₃-C₆ for branched chain). Likewise, cycloalkenyl groups may have from five to eight carbon atoms in their ring structure, and in one embodiment, cycloalkenyl groups have five or six carbons in the ring structure. The term “C₂-C₆” includes alkenyl groups containing two to six carbon atoms. The term “C₃-C₆” includes alkenyl groups containing three to six carbon atoms.

“Heteroalkenyl” includes alkenyl groups, as defined herein, having an oxygen, nitrogen, sulfur or phosphorous atom replacing one or more hydrocarbon backbone carbons.

The term “substituted alkenyl” refers to alkenyl moieties having substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), branched alkynyl groups, and cycloalkyl or cycloalkenyl substituted alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

“Heteroalkynyl” includes alkynyl groups, as defined herein, having an oxygen, nitrogen, sulfur or phosphorous atom replacing one or more hydrocarbon backbone carbons.

The term “substituted alkynyl” refers to alkynyl moieties having substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aryl” includes groups with aromaticity, including “conjugated”, or multicyclic, systems with at least one aromatic ring. Examples include phenyl, benzyl, etc.

“Heteroaryl” groups are aryl groups, as defined above, having from one to four heteroatoms in the ring structure, and may also be referred to as “aryl heterocycles” or “heteroaromatics”. As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=one or two). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthridine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The aryl or heteroaryl aromatic ring can be substituted at one or more ring positions with such substituents as described above, for example, alkyl, alkenyl, akynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl).

As used herein, “carbocycle” or “carbocyclic ring” is intended to include any stable monocyclic, bicyclic or tricyclic ring having the specified number of carbons, any of which may be saturated, unsaturated, or aromatic. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” includes any ring structure (saturated or partially unsaturated or aromatic) which contains at least one ring heteroatom (e.g., N, O or S). Examples of heterocycles include, but are not limited to, morpholine, pyrrolidine, tetrahydrothiophene, piperidine, piperazine and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5 (4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted”, as used herein, means that any one or more hydrogen atoms on the designated atom is replaced with a selection from the indicated groups, provided that the designated atom's normal valency is not exceeded, and that the substitution results in a stable compound. When a substituent is keto or oxo (i.e., —O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R₁) occurs more than one time in any constituent or formula for a compound, its definition at each occurrence is independent of its definition at every other occurrence. Thus, for example, if a group is shown to be substituted with 0-2 R₁ moieties, then the group may optionally be substituted with up to two R₁ moieties and R₁ at each occurrence is selected independently from the definition of R₁. Also, combinations of substituents and/or variables are permissible, but only if such combinations result in stable compounds.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” or “carboxy” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

“Acyl” includes moieties that contain the acyl radical (—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include benzoyl, naphthoyl, etc.

“Alkoxyalkyl”, “alkylaminoalkyl” and “thioalkoxyalkyl” include alkyl groups, as described above, wherein oxygen, nitrogen or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen atom bonded to two carbon atoms or heteroatoms. For example, the term includes “alkoxyalkyl”, which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls and alkthioalkynyls. The term “alkthioalkyls” include moieties with an alkyl, alkenyl or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” includes moieties where a nitrogen atom is covalently bonded to at least one carbon or heteroatom. “Alkylamino” includes groups of compounds wherein nitrogen is bound to at least one alkyl group. Examples of alkylamino groups include benzylamino, methylamino, ethylamino, phenethylamino, etc.

“Dialkylamino” includes groups wherein the nitrogen atom is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively.

“Alkylarylamino”, “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

The term “amidine” or “amidinyl” includes compounds or moietyies having the general structure of —C(═NR)NR′R″, N(R′R″)—CR(═N)—, or CR′(═NR)NR″—, in which R, R′, and R″ can each independently be H, alkyl, cycloalkyl, cycloalkenyl, heterocycle, aryl, or heteroaryl, etc. One example of amidinyl is —C(═NH)NH₂. Amidines can be substituted with substituents such as straight chain alkyl, branched alkyl, cycloalkyl, aryl, heteroaryl or heterocycle. Substituents on amide groups may be further substituted.

Compounds of the present invention that contain nitrogens can be converted to N-oxides by treatment with an oxidizing agent (e.g., 3-chloroperoxybenzoic acid (m-CPBA) and/or hydrogen peroxides) to afford other compounds of the present invention. Thus, all shown and claimed nitrogen-containing compounds are considered, when allowed by valency and structure, to include both the compound as shown and its N-oxide derivative (which can be designated as N→O or N⁻—O⁻). Furthermore, in other instances, the nitrogens in the compounds of the present invention can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present invention includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present invention. Furthermore, so-called metabolite which is produced by degradation of the present compound in vivo is included in the scope of the present invention.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four nonidentical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this invention include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine.

It is to be understood that the compounds of the present invention may be depicted as different tautomers. It should also be understood that when compounds have tautomeric forms, all tautomeric forms are intended to be included in the scope of the present invention, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure and are substituted with various groups as described herein. For example, all of the compounds represented by Formula I are 10,17-dihydroxyl DHA derivatives and have Formula I as a common core.

The present invention is intended to include all isotopes of atoms occurring in the present compounds. Isotopes include those atoms having the same atomic number but different mass numbers. By way of general example and without limitation, isotopes of hydrogen include protium, tritium and deuterium, and isotopes of carbon include C-13 and C-14.

“Pharmaceutically acceptable excipient” means an excipient that is useful in preparing a pharmaceutical composition that is generally safe, and includes any excipient that is acceptable for veterinary use as well as human pharmaceutical use. A “pharmaceutically acceptable excipient” as used in the specification and claims includes both one and more than one such excipient.

“Protecting group”: as used herein, means that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, certain exemplary oxygen protecting groups may be utilized. These oxygen (or hydroxyl) protecting groups include, but are not limited to methyl ethers, substituted methyl ethers (e.g., MOM (methoxymethyl ether), MTM (methylthiomethyl ether), BOM (benzyloxymethyl ether), and PMBM (p-methoxybenzyloxymethyl ether)), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, and TBDPS (t-butyldiphenyl silyl ether), esters (e.g., formate, acetate, benzoate (Bz), trifluoroacetate, and dichloroacetate), carbonates, cyclic acetals and ketals, and glycol ethers, such as ethylene glycol and propylene glycol derivatives and allyl ethers. In certain other exemplary embodiments, nitrogen protecting groups are utilized. Nitrogen protecting groups, as well as protection and deprotection methods are known in the art. Nitrogen protecting groups include, but are not limited to, carbamates (including methyl, ethyl and substituted ethyl carbamates (e.g., Troc), amides, cyclic imide derivatives, N-Alkyl and N-Aryl amines, imine derivatives, and enamine derivatives, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitroveratryloxycarbonyl (“NVOC”) and the like. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

### Synthesis of 10,17-Dihydroxyl DHA Compounds

The present disclosure provides methods for the synthesis of the compounds of Formula I-IV.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, ester or prodrug thereof.

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present invention.

Compounds of the present invention can be conveniently prepared by a variety of methods familiar to those skilled in the art. The compounds of this invention with Formula I-IV may be prepared according to the following Scheme from commercially available starting materials or starting materials which can be prepared using literature procedures. These procedures show the preparation of representative compounds of this invention.

### Scheme 1

One general procedure is illustrated below.

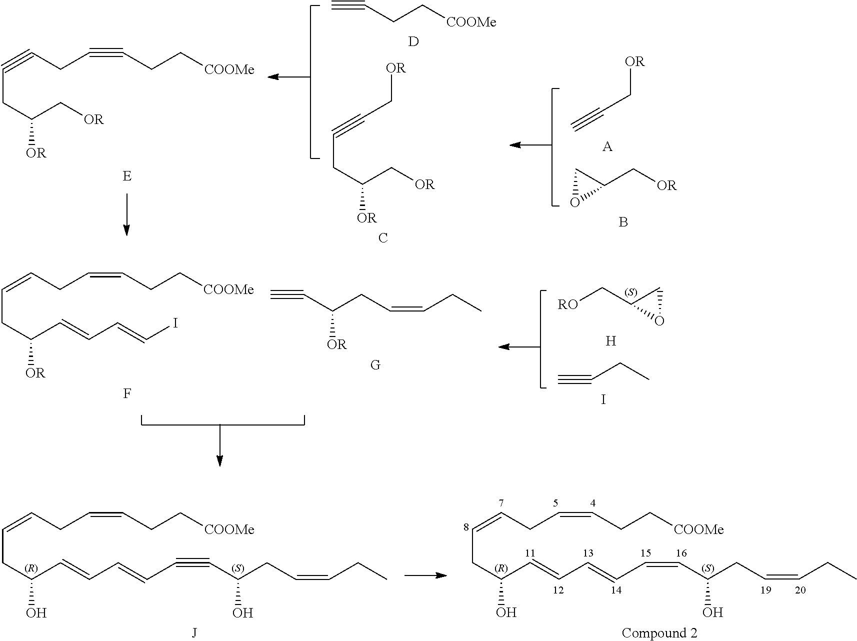

Scheme 1 above demonstrates strategy for total synthesis of Compound 2 and related isomers. The C-10 and C-17 stereochemistry of Compound 2 is derived from enantiomerically pure glycidol derivatives B and H which are reacted with alkynyl nucleophiles derived from A and I, respectively. The (Z) alkene geometry at positions 4-5, 7-8, 15-16 and 19-20 was obtained from selective hydrogenation of acetylenic precursors, which were constructed using coupling procedures. The (E) geometry at positions 11-12 and 13-14 was secured during the synthesis of intermediate F. Other stereoisomers of Compound 2 can be synthesized similarly.

Each of the stereocontrolled steps from defined precursors enabled preparation of geometric isomers of the conjugated triene region that were confirmed by NMR (see US Publication No. 2009/0156673).

### 2. Methods of Treatment

In one embodiment the compositions are suitable for the treatment or prophylaxis of hearing or balance disorders.

The present invention provides methods for slowing the progression or the prevention of at least one symptom or sign of a hearing or balance disorder in a subject in need thereof by administering to a subject in need of such treatment, a therapeutically effective amount of a compound chosen from Table 1 formulated in a composition described herein.

The present invention provides methods for restoring hearing in a subject with clinically defined hearing loss by administering to a subject in need of such treatment, a therapeutically effective amount of a compound chosen from Table 1 formulated in a composition described herein.

The articles “a” and “an” are used herein to refer to one or to more than one (e.g., to at least one) of the grammatical object of the article. By way of example, “an element” means one element or more than one element.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds. The therapeutic agents may be administered in a sequential manner or simultaneous manner. Substantially simultaneous administration can be accomplished, for example, by administering to the subject a single vial having a fixed ratio of each therapeutic agent or in multiple, single vials for each of the therapeutic agents. Sequential or substantially simultaneous administration of each therapeutic agent can be effected by any appropriate route including, but not limited to, topical ocular instillation, oral routes, intravenous routes, intramuscular routes, and direct absorption through mucous membrane tissues, as by intranasal administration. The therapeutic agents can be administered by the same route or by different routes. For example, a first therapeutic agent of the combination selected may be administered by topical ocular instillation while the other therapeutic agents of the combination may be administered orally. Alternatively, for example, all therapeutic agents may be administered by topical ocular administration or all therapeutic agents may be administered by subconjunctival, subscleral, oral or intravenous injection. The therapeutic agent may be administered with other biologically active ingredients and non-drug therapies (e.g., surgery or physical therapy).

As used herein, the term “subject” includes any human or non-human animal. For example, the methods and compositions described herein can be used to treat a subject having sepsis. In certain embodiments provided herein the subject is a human. The term “non-human animal” includes all vertebrates, e.g., mammals and non-mammals, such as non-human primates, sheep, dog, cow, chickens, amphibians, reptiles, etc.

The term “treating” includes prophylactic and/or therapeutic treatments. The term “prophylactic or therapeutic” treatment is art-recognized and includes administration to a subject of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

A hearing or balance disorder may be any of following: sensorineural hearing loss of any cause, age-related hearing loss, noise-induced hearing loss, drug-induced hearing loss, tinnitus, Meniere's disease, vertigo.

Treatment of a subject in need thereof may be by administration through injection of a pharmaceutical composition of the invention into the middle ear cavity to be deposited for continued release of a compound chosen from Table 1 in a therapeutically effective amount into the inner ear over defined period of time.

Duration of release of the compound from the middle ear into the inner ear may depend on the condition to be treated or prevented and may last for 1 week, 2 weeks, 3 weeks, 4 weeks or even longer.

A composition of the present invention for the treatment, prevention or restoration hearing loss may be administered as a combination of the selected compound with a second active therapeutic agent to treat a balance or hearing disorder. Second active therapeutic agents in the treatment of hearing and balance disorders include but are not limited to tropomyosin receptor kinase agonists, canonical growth factors, nerve growth factor, fibroblast growth factor-1-10, neurotrophin-3, brain-derived neurotrophic factor, platelet-derived growth factor, other neurotrophic factors, PI3K/Akt pathway activators, glycogen synthase kinase 3 beta inhibitors, nogo-A inhibitors, HDAC inhibitors, gamma secretase inhibitors, notch inhibitors other than gamma secretase inhibitors including antibodies directed against notch-A and notch-decoy proteins.

In another embodiment the compositions are suitable for the treatment or prevention of ocular disorders.

The present invention provides methods for slowing the progression or the prevention of at least one symptom or sign of an ocular disorder in a subject in need thereof by administering to a subject in need of such treatment, a therapeutically effective amount of a compound chosen from Table 1 formulated in a composition described herein.

The ocular disorder may be a retinal disease including but not limited to age-related macular degeneration of both wet and dry forms, retinal vein occlusion, diabetic macular edema, diabetic retinopathy, all forms of retinitis pigmentosa, Stargardt's disease, retinopathy of prematurity.

Treatment of a subject in need thereof may be by administration through injection of a pharmaceutical composition of the invention into the vitreous to be deposited for continued release of a compound chosen from Table 1 in a therapeutically effective amount to reach the retina over defined period of time.

Duration of release of the compound into the vitreous may depend on the condition to be treated or prevented and may last for 1 month, 2 months, 3 months, 4 months or even longer.

A composition of the present invention may be administered as a combination of the selected compound with a second active therapeutic agent to treat an ocular disease. Second active therapeutic agents in the treatment of ocular disease include but are not limited to anti-nitric oxide synthase inhibitors, antioxidants, anti-inflammatory agents, nrf2 activators, rho kinase inhibitors, anti-vascular endothelial growth factor inhibitors.

As used herein, the term “slowing of progression“ ” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Therapeutically effective dosages are expected to decrease the severity of a sign or symptom.

As used herein the term “symptom” is defined as an indication of disease, illness, injury, or that something is not right in the body. Symptoms may be felt or noticed by the individual experiencing the symptom, but may primarily be noticed by health-care professionals. Others, defined as non-health-care professionals may also observe symptoms.

In general, a suitable daily dose of the compound selected from Table 1, alone or in combination, to be released from the deposited composition will be that amount of the compound(s) which is the lowest dose effective to produce a therapeutic effect. Such an effective dose will generally depend upon the factors described above. Generally, transtympanic or intravitreal doses of the compounds of this invention for a patient, when used for the indicated therapeutic effects, will range from about 0.000001 to about 100 mg per kilogram of body weight per day, more preferably from about 0.00001 to about 1 mg per kg per day, and still more preferably from about 0.0001 to about 0.01 mg per kg per day.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Examples

### Example 1: Copolymer (PEG-PCL-PEG) Synthesis of Drug Formulation Intended for Drug Delivery Through Controlled Diffusion

### Method:

0.098M &-caprolactone and 0.007M MPEG was added into a three necked vessel under a dry nitrogen atmosphere. Stannous octate (100 μL) was added and maintained at the reaction temperature of 125° C. for 24 hours with isophorone diisocyanate (IPDI) (0.008M) added as a coupling agent at the 6 hr time point while stirring. The resultant copolymer was degassed for 10 min. To isolate the pure copolymer, the product was cooled to room temperature, dissolved in dichloromethane and subsequently precipitated with petroleum ether which was previously refrigerated for 1 hour. The precipitate was then filtered and vacuum dried at room temperature for 24 hours. An aqueous solution of compound 1 was produced at varying concentrations (1 mg/mL and 2 mL total volume). The compound 1 solution (0.1% wv) was added to a 30% w/v copolymer solution to produce a physical homogenous blend.

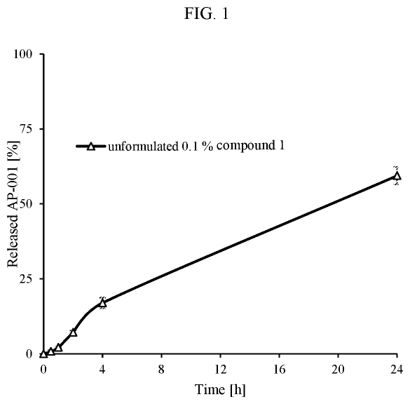

### Example 2: Release Profile of Compound 1 Formulated in an Artificial Tear Medium Over 24 Hours (FIG. 1)

### Method:

0.5 mL of unformulated compound 1 dissolved in artificial tear medium (i.e., equivalent to physiological buffer solution/PBS) was filled into dialysis tubes (Spectra/Por® Biotech CE MWCO 100-500, 10 mm), sealed and immersed into containers filled with 5 mL artificial tear fluid (ATF) serving as release medium (Table 2). The samples were incubated at room temperature and shaken at 100 rpm. After 0 hours, 0.5 hours, 1 hour, 2 hours, 4 hours and 24 hours, samples were withdrawn and the amount of released compound 1 was quantified via HPLC.

TABLE 2
Time points and release (%) of compound 1 over 24 hours

| Time points (h) | Release (%) |
|---|---|
| 0.5 | 0.8 |
| 1 | 2 |
| 2 | 7 |
| 4 | 17 |
| 24 | 59 |

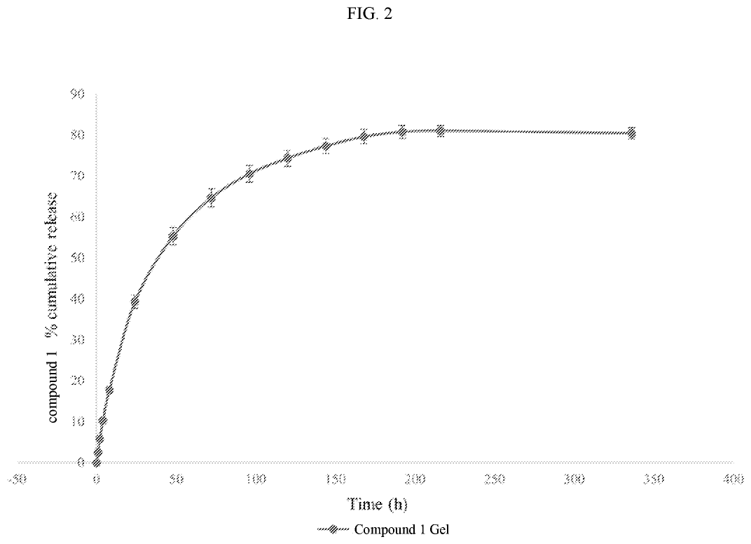

### Example 3: Release Profile of Compound 1 Formulated in the PEG-PCL-PEG Co-Polymer (FIG. 2)

### Formulation:

Compound 1 was loaded into a thermo-responsive gel at 1 mg/mL concentration. Compound 1 solution (0.1% w/v) was added to a 30% w/v copolymer solution described in Example 1 to produce a physical homogenous blend.

### Drug Release:

Briefly, 0.5 mL of compound 1 gel was added into a dialysis bag (MWCO 14,000 Da). The sample loaded tubings were sealed and placed in 30 mL glass bottles containing 20 mL of PBS 7.4 as release medium and incubated at 37±0.5° C. under shaking condition at 50 rpm. 3 mL samples were withdrawn at specific time intervals from the bottles and an equal amount of fresh PBS was added to maintain a constant volume and sink condition. The study was performed in triplicate. The prepared gel showed sustained release of the loaded compound 1 over 8 days.

The formulation prepared in Example 3 of compound 1 formulated with the copolymer of Example 1 is also referred to herein as the triblock formulation.

In vitro data showing a long release correlates to sustained release in vivo, as supported by Wang et al. Laryngoscope 121:385-391, 2011, which is incorporated herein by reference.

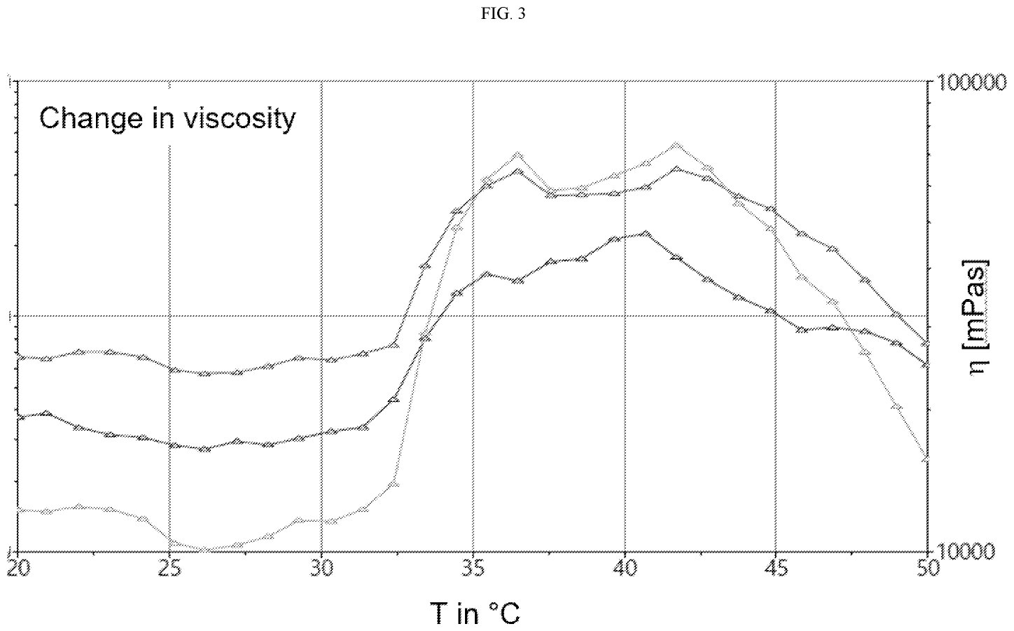

### Example 4: Liquid to Semi-Solid Temperature-Dependent Conversion (FIG. 3)

### Gelling Time:

In a separate experiment on increasing gel temperature from room temperature to body temperature the co-polymer was shown to gel at ˜9 minutes with no further increase in gelling at higher temperature. The obtained consistency was that of a viscous gel and not semi-solid to solid in consistency. The gelling was reversible: on reducing temperature the increase in viscosity was lost.

### Example 5: In Vivo Assessment of Triblock Gel for Transtympanic Delivery of Drug to the Middle Ear

### Materials and Animals

Wistar rat at 225±25 grams.

### Injection:

Transtympanic injection into the middle ear cavity

### Material and Volume:

Dried, powdered triblock formulation of Example 3 was reconstituted in water to a 30% w/v formulation as intended for clinical use. Reconstituted formulation was observed liquid at room temperature and confirmed gelling to viscous consistency at 37° C. within 10 minutes. A volume of 30 mL was injected into each ear.

### Experimental Design:

Nine (9) animals were injected with the formulation at timepoint 0 and then euthanized at various times thereafter for inspection of retention of gelled formulation in the middle ear. Injections were done bilaterally. Post injection timepoints: 0.5/1 hours, 3 days, and 7 days with 3 animals (6 ears) per timepoint.

### Outcome:

Primary outcome was visual confirmation of gelled formulation present in the middle ear by photographic confirmation. All ears except one, at the 3-hour timepoint, showed retained gel coating relevant areas around the round window which is the target membrane for drug diffusion from middle ear into the inner ear, the therapeutic purpose of delivering drugs using the triblock formulation.

### INCORPORATION BY REFERENCE

All publications, patents, and patent applications mentioned herein are hereby incorporated by reference in their entirety as if each individual publication, patent, or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. An controlled release pharmaceutical composition containing an effective amount of a compound of Formula I, II, III or IV: 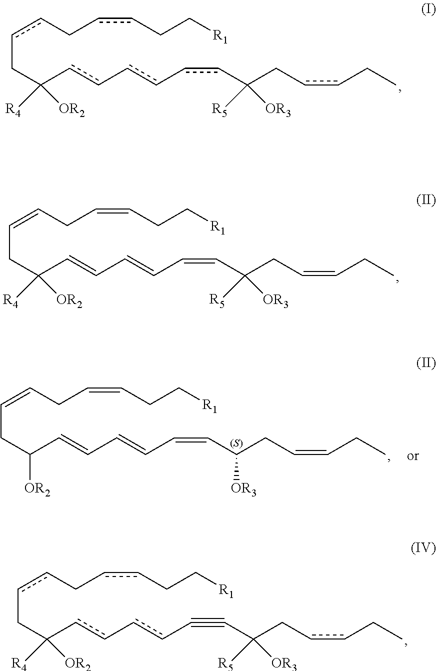
wherein [symbol]
represents a cis or trans bond;
R₁ is —C(O)ORₐ, —C(O)NR_{b}R_{c}, —C(O)H, —C(NH)NR_{b}R_{c}, —C(S)H, —C(S)ORₐ, —C(S)NR_{b}R_{c}, or —CN;
R₂ and R₃ are each independently H or a protecting group;
R₄ and R₅ are each independently H, halo, unsubstituted or substituted C₁-C₆ alkyl, unsubstituted or substituted C₃-C₁₀ carbocycle, unsubstituted or substituted heterocycle comprising one or two 3-, 4-, 5- or 6-member rings and 1-4 heteroatoms selected from N, O and S,
Rₐ is a protecting group or -T₁-Q₁;
R_{b} and R_{c} are each independently a protecting group or -T₁-Q₁, or R_{b} and R_{c}, together with the nitrogen atom to which they are attached, form an unsubstituted or substituted heterocycle comprising one or two 5- or 6-member rings and 1-4 additional heteroatoms selected from N, O and S;
T₁ is a bond or unsubstituted or substituted C₁-C₆ alkyl linker; and
Q₁ is H, hydroxyl, halogen, unsubstituted or substituted C₁-C₆ alkyl, unsubstituted or substituted C₁-C₆ alkoxy, unsubstituted or substituted 2-6 membered heteroalkyl, unsubstituted or substituted C₃-C₁₀ carbocycle, or unsubstituted or substituted heterocycle comprising one or two 3-, 4-, 5- or 6-member rings and 1-4 heteroatoms selected from N, O and S,
or a pharmaceutically acceptable salt, ester, or prodrug thereof.

2. A method of treating or preventing a disease or condition comprising administering the controlled release pharmaceutical composition of claim 1, and the disease or condition is a corneal disorder, a retinal disorder, a balance disorder, or a hearing disorder.

3. The method of claim 2, wherein the pharmaceutical composition comprises a pharmaceutically acceptable excipient.

4. The method of claim 2 or 3, wherein said pharmaceutically acceptable excipient comprises one or more of:
a. PEG: polyethylene glycol;
b. PLC: poly (ε-caprolactone);
c. PGA: polyglycolic acid;
d. PLGA: poly (lactic-co-glycolide)

5. The method of claim 3 or 4, wherein said pharmaceutically acceptable excipient comprises tri-block or penta-block polymers.

6. The method of claim 5, wherein the selection of or sequence of PEG, PLC, PGA, and PLGA in creating a tri-block or a penta-block may vary.

7. The method of claim 5, wherein the pharmaceutical composition comprises the compound encapsulated in a nanoparticle admixed with said tri-block or penta-block polymers.

8. The method of claim 5, wherein said tri-block or penta-block polymers are in solution at ambient temperature but at a temperature approximately that of body temperature form a gel, suspension, or emulsion.

9. The method of any one of claims 2-8, wherein the pharmaceutical composition releases the compound at a therapeutically effective daily dose over 1, 2, 3, or 4 weeks, or longer for up to 6 months after administration.

10. The method of any one of claims 2-9, wherein the pharmaceutical composition is retained in the middle ear for 7 or more days after injection into the middle ear cavity.

11. The method of any one of claims 2-10, wherein the pharmaceutical composition comprises a second active agent.

12. The method of any one of claim 2-11, wherein treatment of the condition results in amelioration of the condition and restoration towards normal of a pre-existing condition.

13. The method of any one of claims 2-12, wherein the condition is a hearing disorder or balance disorder.

14. The method of any one of claims 2-12, wherein the condition is a retinal disorder.

15. The method of any one of claims 2-12, wherein the condition is a corneal disorder.

16. The method of claim 13, wherein the pharmaceutical composition is delivered via transtympanic injection to the inner ear.

17. The method of claim 14 or 15, wherein the pharmaceutical composition is delivered via injection to the vitreous.

18. The method of claim 14 or 15, wherein the pharmaceutical composition is delivered as an eye drop or eye ointment to the ocular surface.

19. The method of any one of claims 2-18, wherein the compound is any one of the compounds listed in Table 1 or a salt thereof.

20. The method of any one of claims 2-18, wherein the compound is sodium (4Z,7Z, 10R, 11E, 13E, 15Z, 17S, 19Z)-10, 17-dihydroxydocosa-4,7,11, 13, 15, 19-hexaenoate or (4Z,7Z, 10R, 11E, 13E, 15Z, 17S, 19Z)-10, 17-dihydroxydocosa-4,7,11, 13, 15, 19-hexaenoic acid.

21. The method of any one of claims 2-20, wherein the compound is administered at a dosage from about 0.0001-0.1 mg per kg per day.

22. A method of making the controlled release pharmaceutical composition of claim 1, wherein the pharmaceutical composition comprises tri-block or penta-block polymers, and the compound of Formula I, II, II, IV is dissolved in propylene glycol prior to adding to the tri-block or penta-block polymers.

23. A method of making the controlled release pharmaceutical composition of claim 1, wherein the pharmaceutical composition comprises tri-block or penta-block polymers, and the compound of Formula I, II, II, IV is dissolved in ethanol prior to adding to the tri-block or penta-block polymers.
